SEQUENCE LISTING (12 sequences)

SEQ ID NO: 1 - DNA, 20 bp, Artificial Sequence
  Synthetic primer
caacttgatccacgttcacc

SEQ ID NO: 2 - DNA, 20 bp, Artificial Sequence
  Synthetic primer
gaagagccaaggacaggtac

SEQ ID NO: 3 - DNA, 19 bp, Artificial Sequence
  Synthetic primer
ctaggtctaagcgaccttg

SEQ ID NO: 4 - DNA, 19 bp, Artificial Sequence
  Synthetic primer
tctggtctctgtagagttc

SEQ ID NO: 5 - DNA, 24 bp, Artificial Sequence
  Synthetic primer
ccttatctacgtggtcattgatct

SEQ ID NO: 6 - DNA, 20 bp, Artificial Sequence
  Synthetic primer
aagccactgctctgtacctg

SEQ ID NO: 7 - DNA, 20 bp, Artificial Sequence
  Synthetic primer
tgttggcttccccttcatct

SEQ ID NO: 8 - DNA, 20 bp, Artificial Sequence
  Synthetic primer
tggagcggaagttgtcgtat

SEQ ID NO: 9 - DNA, 22 bp, Artificial Sequence
  Synthetic primer
tatgatgacatcaagaaggtgg

SEQ ID NO: 10 - DNA, 18 bp, Artificial Sequence
  Synthetic primer
atgtcgttgtcccaccac

SEQ ID NO: 11 - DNA, 20 bp, Artificial Sequence
  Synthetic primer
cgtagcgatagtcttagcac

SEQ ID NO: 12 - DNA, 20 bp, Artificial Sequence
  Synthetic primer
gtgccacaccaaaactacag

(19) United States
(12) Patent Application Publication
(10) Pub. No.: US 2010/0184725 A1
(43) Pub. Date: Jul. 22, 2010

(54) Method and Composition for Treating Alzheimer's Disease and Dementias of Vascular Origin

(75) Inventor: Anil Gulati, Naperville, IL (US)

Correspondence Address: MARSHALL, GERSTEIN & BORUN LLP, 233 SOUTH WACKER DRIVE, 6300 WILLIS TOWER, CHICAGO, IL, 60606-6357, US

(73) Assignee: THE BOARD OF TRUSTEES OF THE UNIVERSITY OF ILLINOIS, Urbana, IL (US)

(21) Appl. No.: 12/698,239

(22) Filed: Feb. 2, 2010

Related U.S. Application Data
(62) Division of application No. 10/659,579, filed on Sep. 10, 2003.
(60) Provisional application No. 60/413,539, filed on Sep. 25, 2002.

(51) Int. Cl. A61K 31/66; A61K 31/55; A61K 31/5377; A61K 31/502; A61K 31/513; A61K 31/4353; A61K 31/445; A61P 25/28

(52) U.S. Cl. 514/129; 514/215; 514/235.8; 514/249; 514/269; 514/297; 514/317

(57) ABSTRACT

A composition and method of treating Alzheimer's disease or a dementia of vascular origin are disclosed. The composition and method utilize an endothelin antagonist as the active agent to treat Alzheimer's disease or a dementia of vascular origin in mammals, including humans.

### CROSS-REFERENCE TO RELATED APPLICATION

This application claims the benefit of U.S. provisional patent application No. 60/413,539, filed Sep. 25, 2002.

### FIELD OF THE INVENTION

The present invention relates to the treatment of Alzheimer's disease and dementias of vascular origin, in a mammal, by administration of a therapeutically effective amount of an endothelin antagonist.

### BACKGROUND OF THE INVENTION

The fastest growing segment of the U.S. population is individuals aged 65 years or older (Yancik, 1997). As a result of this demographic shift, the number of individuals aged 75 years is expected to triple, and the number of individuals over 85 years to double, over the next 30 years (Yancik, 1997). Aging is associated with progressive deterioration of the normal functions of an individual, in particular a decline in the function of the central nervous system (CNS). Aging causes a decrease in the brain functions in the geriatric population, resulting in impaired or hampered motor activities, and compromises the quality of life.

A variety of degenerative diseases of the brain, including Alzheimer's disease (AD), are associated with aging. Brain aging is characterized by neuronal cell loss mostly in the caudate putamen and subcortical cholinergic nuclei (Coleman et al., 1987(a); Coleman et al., 1987(b)). Cardiovascular and cerebrovascular diseases also are the leading causes of death in the elderly (Cohen, 1997). It also has been established that aging is one of the most potent risk factors for cerebrovascular injuries (Starr et al., 1993). In the vasculature, aging is associated with modest thickening of wall elements and functional changes of vascular smooth muscle and endothelium (Luscher et al., 1992). The structural remodeling of intracerebral arterial vessels during aging, action of different vasoactive factors, and rheological disturbances all can interfere with local flood flow in this disease and cause parenchymal changes in the brain tissue during aging.

Aging is an important risk factor for AD. Low education and hypertension also are known risk factors for AD. Epidemiological data has identified hypertension and stroke as the most potent risk factors for the development of a vascular dementia. However, the differential diagnosis between AD and vascular dementia remains clinically challenging.

The most prominent feature of AD is the presence of extracellular neuritic plaques, which have β-amyloid (Aβ) at their core. Aβ is cleaved from the amyloid precursor protein (APP). It has been theorized that Aβ has a significant vasoactive role. Therefore, increasing concentrations of Aβ can contribute to AD pathology by inducing microvascular vasoconstriction and reducing cerebral blood flow, which results in hypoperfusion and ischemia.

It has been shown that Aβ can enhance vasoactivity induced by endothelin-1 (ET-1), a potent cerebrovascular vasoconstrictor. Increased amounts of ET-1-type immunoreactivity has been observed in astrocytes obtained from AD patients. Research also indicates that ET-1 is an important neuromodulator in the central nervous system (CNS). ET-1, therefore, can act as a neuromodulator by causing a severe reduction of the cerebral blood flow.

### SUMMARY OF THE INVENTION

The present invention is directed to administration of an endothelin antagonist in the treatment of Alzheimer's disease (AD) and dementias of vascular origin.

Accordingly, one aspect of the present invention is to provide a method of treating Alzheimer's disease or a dementia of vascular origin comprising administering to a mammal in need thereof a therapeutically effective amount of an endothelin antagonist. The endothelin antagonist can be an endothelin A antagonist, an endothelin B antagonist, or a mixed endothelin A/B antagonist.

Another aspect of the present invention is to provide a composition comprising an endothelin antagonist. The composition is useful in the treatment of AD and dementias of vascular origin. In particular, the present invention is directed to compositions containing an endothelin antagonist, and to methods of administering the composition to treat AD and dementias of vascular origin.

Another aspect of the present invention is to provide a composition comprising an endothelin antagonist, a second therapeutic agent useful in the treatment of AD or a dementia of vascular original, and an excipient.

Still another aspect of the present invention is to provide an article of manufacture for human pharmaceutical use, comprising (a) a container, (b1) a packaged composition comprising an endothelin antagonist and, optionally, (b2) a packaged composition comprising a second therapeutic agent useful in the treatment of AD or a dementia of vascular origin, and (c) a package insert containing directions for use of the composition or compositions, administered simultaneously or sequentially, in the treatment of AD or a dementia of vascular origin.

These and other aspects of the present invention will become apparent from the following detailed description of the preferred embodiments taken in conjunction with the figures.

### BRIEF DESCRIPTION OF THE DRAWINGS

FIG. 1 is a flow chart showing the relationship between aging, hypertension, APP, Aβ, endothelin, and dementia;

FIG. 2 contains bar graphs showing the effect of Aβ on brain blood flow (ml/min/100 g) in various regions of the brain;

FIG. 3 contains bar graphs showing the effect of Aβ on systemic hemodynamics;

FIG. 4 contains plots of blood flow (ml/-min/100 g) vs. time showing the effect of ET-1 infusion in control and Aβ-treated rats; and

FIG. 5 contains plots showing the results of ET-1 mRNA expression in control (saline) and Aβ-treated rats by semiquantitative RT-PCR, and contains bar graphs showing the relative intensity (ET-1/GAPDH) in various plots of a rat brain.

### DETAILED DESCRIPTION OF THE PREFERRED EMBODIMENTS

The present invention is directed to compositions and methods of treating Alzheimer's disease (AD) and dementias of vascular origin. The present invention also is directed to pharmaceutical compositions comprising an endothelin antagonist and a second therapeutic agent useful in the treatment of AD or a dementia of vascular origin. Further provided are articles of manufacture comprising an endothelin antagonist and, optionally, a second therapeutic agent useful in the treatment of AD or a dementia of vascular origin, packaged separately or together, and an insert having instructions for using these active agents.

The methods described herein benefit from the use of an endothelin antagonist and an optional second therapeutic agent in the treatment of AD or a dementia of vascular origin in the treatment and management of AD or a dementia of vascular origin. The endothelin antagonist and an optional second therapeutic agent in the treatment of AD or a dementia of vascular origin can be administered simultaneously or sequentially to achieve the desired effect.

For the purposes of the invention disclosed herein, the term “treatment” includes ameliorating, retarding the progression of, or eliminating AD or a dementia of vascular origin. As such, the term “treatment” includes both medical therapeutic and/or prophylactic administration, as appropriate.

The term “container” means any receptacle and closure therefor suitable for storing, shipping, dispensing, and/or handling a pharmaceutical product.

The term “insert” means information accompanying a pharmaceutical product that provides a description of how to administer the product, along with the safety and efficacy data required to allow the physician, pharmacist, and patient to make an informed decision regarding use of the product. The package insert generally is regarded as the “label” for a pharmaceutical product.

The term “prodrug” means compounds that transform rapidly in vivo to a compound useful in the invention, for example, by hydrolysis. A thorough discussion of prodrugs is provided in Higuchi et al., Prodrugs as Novel Delivery Systems, Vol. 14, of the A.C.S.D. Symposium Series, and in Roche (ed.), Bioreversible Carriers in Drug Design, American Pharmaceutical Association and Pergamon Press, 1987.

### Endothelin in the CNS

ET, a vasoconstrictor peptide (Yanagisawa et al., 1988), contains 21 amino acids with a molecular weight of 2492 Da and shows little homology with other known vasoactive peptides. ET is synthesized as a prepropeptide whose gene expression is induced by several vasoactive substances. The presence of mRNA encoding for the proproform of ET has been demonstrated (Yanagisawa et al., 1988). Arai et al. (Arai et al., 1990) cloned a receptor for ET (ET_{A}) and found that it was highly specific for ET-1 binding. Sakurai et al. (Sakurai et al., 1990) also cloned an ET receptor (ET_{B}) and found that this receptor was nonselective and could not distinguish between ET-1, ET-2, and ET-3 in displacing ET-1.

ET receptors are present in the rat (Bertelsen et al., 1992; Gulati et al., 1991; Iyer et al., 1995), pig (Hensen et al., 1991), and human (Takahashi et al., 1991) brains. Within the CNS, both ET-1 and ET-3 are expressed and present in neurons and glia (Koizumi et al., 1994; Lee et al., 1990; MacCumber et al., 1990). ET mechanisms in the CNS play an important regulatory role in the control of the cerebrovascular system (Gulati et al., 1996; Gulati et al., 1992; Kumar et al., 1996; Rebello et al., 1995; Sagher et al., 1994), behavior (Maslarova et al., 1995), and sensory motor system (Maslarova et al., 1995). ET-converting enzyme activity has been observed in rat brain astrocytes in vitro (Wilkes et al., 1991). The distinct regional distribution of ET receptor binding in the brain suggests that neuronal ET can serve as a neuromodulator or neurotransmitter (Greenberg et al., 1992; Gulati et al., 1995; Gulati et al., 1992). ET receptors in the brain are not restricted to the vascular smooth muscles, but are also associated with neurons, astrocytes, and glial cells (Lee et al., 1990; Yoshizawa et al., 1990). Stimulation of ET_{A} receptors results in severe vasoconstriction of cerebral blood vessels, playing an important role in the regulation of cerebral blood flow (Gulati et al., 1996; Harland et al., 1995; Rebello et al., 1995: Sagher et al., 1994; Yu et al., 1995).

### Endothelin in Age-related CNS Pathological Disorders

Studies have been performed to determine the influence of aging on the central and peripheral ET system. In a study conducted on 16 young (i.e., 25±3 years) and 16 older (i.e., 68±7 years) normal healthy volunteers, it was found that both systemic adrenergic drive and ET-1 levels increased in parallel with aging (White et al., 1997).

It also was found that aging in healthy rodents is associated with a marked upregulation of renal ET-1 protein content, which can promote age-dependent diseases such as glomerulosclerosis, hypertension, and atherosclerosis (Barton et al., 2000). In another study, it was found that vascular ET-1 protein content increases with aging in most of the vascular beds (Goettsch et al., 2001). Further, it was found that aging increases mRNA expression of ET-1 in the heart and that ET-1 mRNA expression is further increased by exercise-induced cardiac hypertrophy (Iemitsu et al., 2002).

Aging also is an important risk factor for AD. The most prominent feature of AD is the extracellular neuritic plaques, which have at their core β-amyloid (Aβ), cleaved from amyloid precursor protein (APP). It has been suggested that Aβ has a significant vasoactive role (Crawford et al., 1998(a)). Increasing concentrations of Aβ can contribute to AD pathology by inducing microvascular vasoconstriction and reducing cerebral blood flow, resulting in hypoperfusion and ischemia (Thomas, 1996).

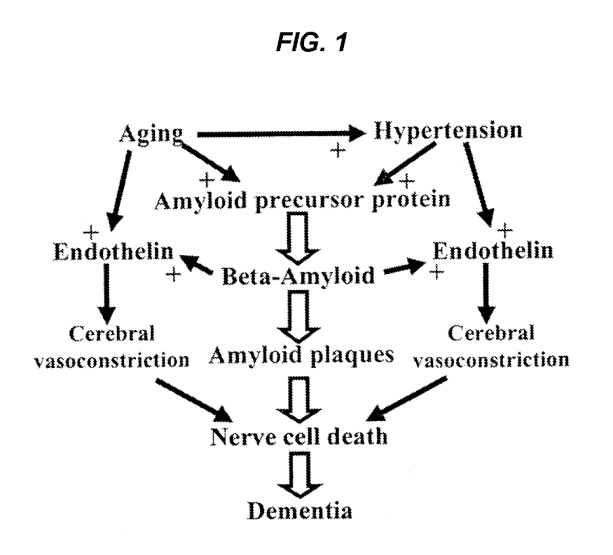

FIG. 1 shows that Aβ has a significant vasoactive role, and that increasing concentrations of Aβ can contribute to AD pathology by inducing release of ET-1, which leads to microvascular vasoconstriction and reduced cerebral blood flow, resulting in hypoperfusion and ischemia.

It has been shown that Aβ can enhance the vasoactivity induced by endothelin-1 (ET-1), a potent cerebrovascular vasoconstrictor (Crawford et al., 1998b). Increased ET-1-like immunoreactivity has been observed in the astrocytes obtained from AD patients (Zhang et al., 1994(a); Zhang et al., 1994(b)). However, in one study, a decrease in ET-1 levels has been observed in the cerebrospinal fluid (CSF) of elderly patients with AD. In this study, it was also found that, no significant difference existed in the CSF ET-1 level among three clinical groups, i.e., disease control, AD, and senile dementia of Alzheimer's type. The number of patients in each group was too low (i.e., 5 to 7) to make any relevant conclusions.

Experiments in vivo illustrated the effect of Aβ on the cardiovascular system, and demonstrated the role of ET. ET-1 and ET-3 have produced astrocyte proliferaton in tissue culture studies (Couraud et al., 1991; Ehrenreich et al., 1991; Hosli et al., 1991; Marin et al., 1991). The increased expression of ET-1-like immunoreactivity by the AD brain astrocytes could explain their proliferation in these disorders (Zhang et al., 1994(a); Zhang et al., 1994(b)). ET-1 can act as a neuromodulator by causing a severe reduction of the cerebral blood flow (Gulati et al., 1996; Kumar et al., 1996; Rebello et al., 1995). A decrease in cerebral blood flow has been confirmed in senile dementia of the Alzheimer's type (Jugust et al., 1987). Decreased ET-1 binding sites have been observed in the cerebral cortex of AD brains, which can be attributed to loss of neurons in the cerebral cortex (Kohzuki et al., 1995).

Several studies have demonstrated the cardiovascular effects of systemically and centrally administered ET-1 in normotensive rats, and have elucidated mechanisms of ET-1 action.

### Cardiovascular Responsiveness to Intracerebroventricular ET-1 in Normotensive Rats: ET_{A}-Mediated and Sympathetic Effects

The systemic hemodynamic and regional circulatory effects of intracerebroventricular administration of ET receptor agonists were investigated in male normotensive Sprague-Dawley rats using an established radioactive microsphere technique (Gulati et al., 1997; Rebello et al., 1995). ET_{A} receptor agonists, ET-1 (30, 45, and 90 ng), ET-2 (90 ng), and sarafotoxin 6b (SRT6b 30 and 90 ng) were administered. ET-1 (45 ng) produced a transient increase (26%) followed by a sustained decrease (48%), accompanied by significant decreases in cardiac output and stroke volume, while total peripheral resistance remained unchanged. Regional blood flows to the brain, heart, kidneys, gastrointestinal, portal, and musculoskeletal systems were reduced between 40% and 75%. The effects of ET-2 were less intense in comparison to those of ET-1 and SRT6b. Pretreatment with the specific ET_{A} receptor antagonist, BQ123 (10 μg ICV), blocked the systemic hemodynamic and regional circulatory effects of ET-1.

In separate experiments, administration of IRL1620, ET-3, and sarafotoxin 6c, all specific ET_{B} receptor agonists, did not affect hemodynamics or regional blood circulation. Thus, it was concluded that ET_{A}, but not ET_{B}, receptors mediate the central effects of ET-1 on cardiovascular function. Furthermore, sympathetic nerve activity was recorded from the left postganglionic splanchnic nerve at its exit from the suprarenal ganglion. Administration of ET-1 (90 ng) significantly decreased sympathetic nerve activity. In a separate study, pretreatment with propranolol blocked the hemodynamic and regional circulatory effects of centrally administered ET-1 (Kumar et al., 1996). These data provide further support that centrally administered ET_{A} receptor agonists mediate their effects through the sympathetic nervous system.

### Changes in Central ET Receptor Density in Hypertensive Rats

Binding of ET-receptor agonists to ET receptors in the cerebral cortex and ventrolateral medulla was studied in 8-week-old spontaneously hypertensive (SHR) and normotensive Wistar-Kyoto (WKY) rats (Gulati, 1991). Both [¹²⁵I]sarafotoxin 6b (SRT6b), a nonspecific ET-receptor agonist, and [¹²⁵I]-ET-1, an ET_{A}-receptor agonist, bound to a single high affinity site. In the cerebral cortex, [¹²⁵I]-SRT6b and [¹²⁵I]-ET-1 demonstrated similar binding characteristics to ET receptors in SHR and WKY rats. In comparison, in the ventrolateral medulla [¹²⁵I]-SRT6b and [¹²⁵I]-ET-1 binding was found to be significantly lower in SHR as compared to WKY rats due to a reduction in receptor binding sites. It, therefore, was concluded that ET receptor density was reduced in the ventrolateral medulla of SHR rats and can contribute to the regulation of blood pressure.

### Changes in Tissue Distribution of ET-1 in a Rodent Model of Spontaneous Hypertension

Plasma and tissue ET-1-like radioimmunoreactivity were measured in a hamster model of spontaneous hypertension, and compared to results obtained in age-matched male normotensive hamsters (Gulati et al., 1998). While plasma ET-1 activity was similar in both groups, renal and cardiac ET-1-like radioimmunoreactivity was 11-fold and 1.7-fold greater in hypertensive hamsters. These data suggest that tissue, rather than plasma, ET-1 can play a role in modulating cardiovascular responses in hypertension (Gulati et al., 1998).

### The Influence of Aging on Central ET Activity

The influence of aging on the binding characteristics of central ET receptors was studied in male Fischer 344 rats aged 4, 15, and 24 months (Bertelsen et al., 1992). ET_{A} receptor density and ET-1 binding affinity in the cerebral cortex and spinal cord were similar in each of the three age groups. ET-2 also showed similar binding characteristics. However, ET-3 demonstrated increased affinity for ET receptors in the spinal cord of 24-month-old rats compared with younger rats in the other two age groups. Because central ET receptors play a role in the regulation of blood pressure (Gulati et al., 1995), the increased sensitivity of ET receptors to ET-3 in the spinal cord of aged rats can contribute to increased predisposition of the elderly to the development of hypertension.

### Effect of Aβ on Systemic Hemodynamics and Regional Blood Circulation

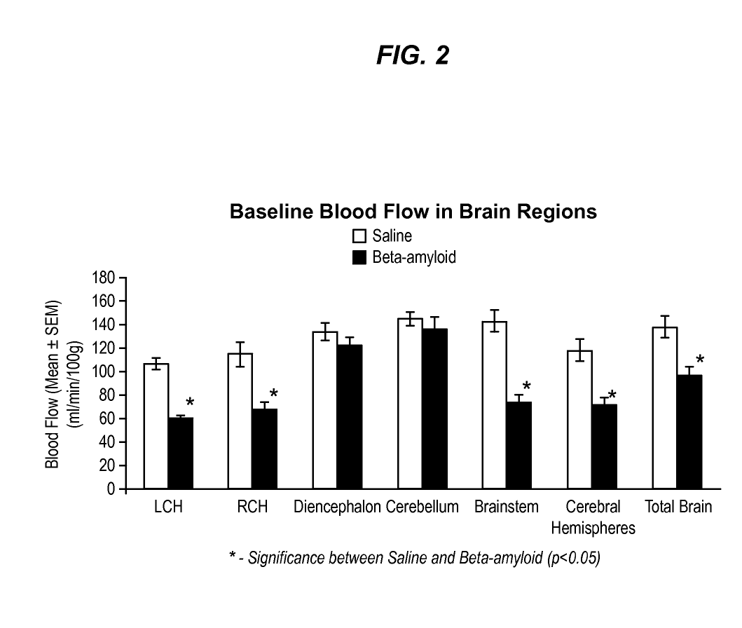

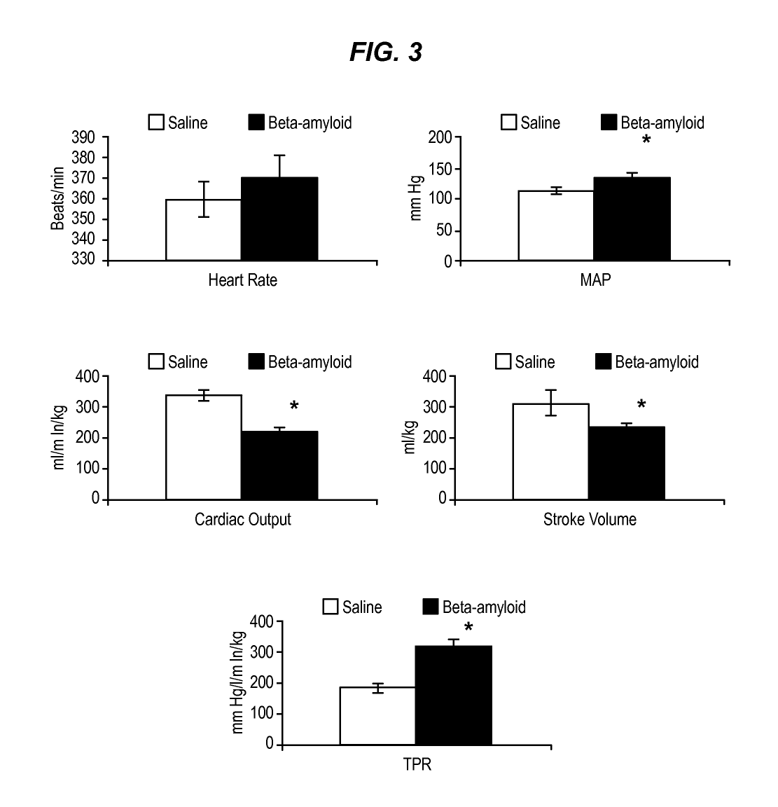

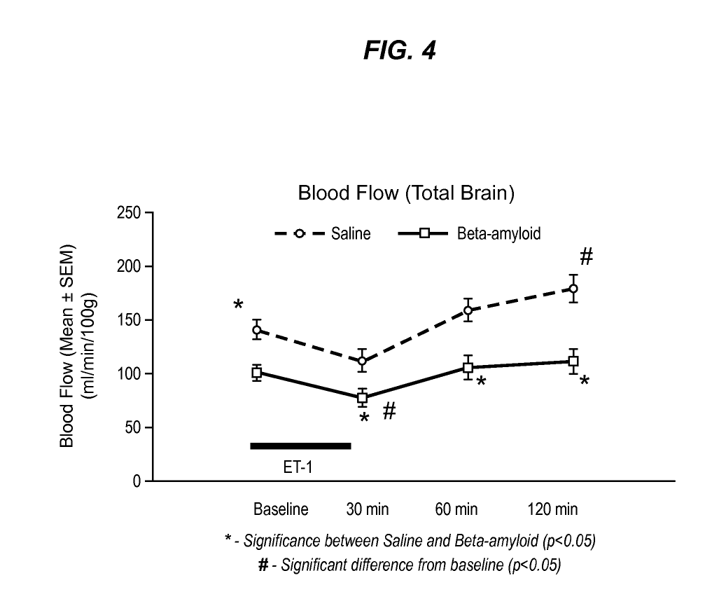

Studies in 3-month-old rats to determine the effect of Aβ on systemic hemodynamics and regional blood circulation were performed. It was found that Aβ produced significant increase in vascular resistance. The results are summarized in FIGS. 2 and 3. The effect of ET-1 on the brain blood flow of control and Aβ treated rats also was determined (FIG. 4). The results show that Aβ is vasoactive, and also alters the response to ET-1.

Effect of Aβ on ET-1 mRNA

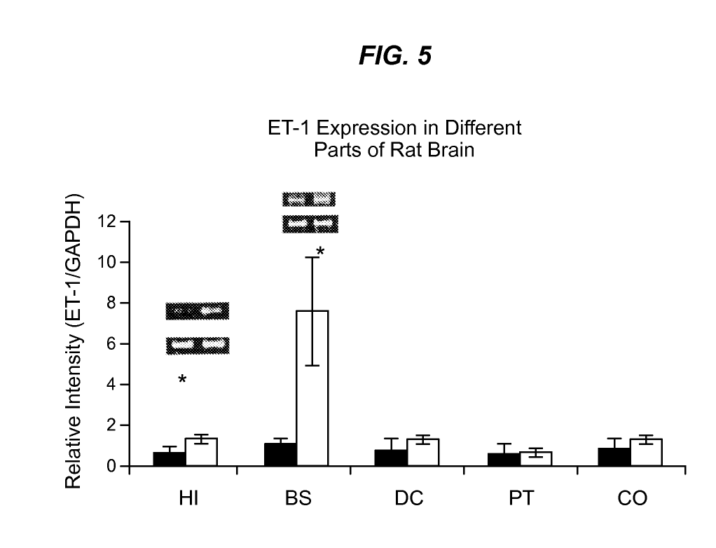

Standardized mRNA expression studies, an example of ET-1 mRNA expression in saline and A13-treated rats, are shown in FIG. 5. It was found that Aβ significantly increased the ET-1 mRNA expression in 3-month-old rats in the hippocampus and brain stem areas of the brain. An increase in ET-1 mRNA expression in brain stem and hippocampus is important because a majority of the cardiovascular regulatory centers are located in brain stem and hippocampus, which is the area most affected in AD.

### Endothelin Antagonists

An endothelin antagonist utilized in the present invention can be any of the endothelin receptor antagonists known in the art. Endothelin is a potent vasoconstrictor. Endothelin antagonists inhibit the activity of endothelin, and are used to treat acute heart failure, congestive/chronic heart failure, pulmonary arterial hypertension, pulmonary edema, subarachnoid hemorrhage, chronic obstructive pulmonary disease, myocardial infarction, acute cerebral ischemia, acute coronary syndromes, acute renal failure, post-operative treatment in liver operations, and prostate cancer.

It has been shown that AD brains show chronic inflammatory responses characterized by activated glial cells and increased expression of cytokines and complement factors surrounding neuritic plaques. A growing body of evidence suggests that these activated glia contribute to neurotoxicity through the induction of inflammatory cytokines, such as interleukin-1 beta (IL-1β) and tumor necrosis factor alpha (TNFα). ET has been shown to participate in the proinflammatory responses. In fact, ET-1 modulates the differentiated state of astrocytes, and the responsiveness of the differentiated astrocytes to ET-1 is due to the extremely high expression of ET_{B} receptor. ET also stimulates cytokine production by macrophages or microglial cells and also is involved in the initiation of gliosis following acute brain damage. These studies indicate that stimulation of ET_{B} receptors located on the astrocytes (glia) could contribute to neurotoxicity through the induction of inflammatory cytokines such as IL-1β and TNFα.

The role of ET and its receptor expression in the brain and their correlation with inflammatory action in AD is possible. Because evidence is accumulating that nonsteroidal antiinflammatory agents are effective in AD, it is theorized, but not relied upon herein, that Aβ causes overexpression of ET-1 and ET_{B} receptor in the brain leading to inflammatory response due to ET_{B} receptor stimulation. Therefore, ET_{A} and ET_{B} receptor antagonists, and balanced ET_{A}/ET_{B}, could be useful in the treatment and/or prevention of AD. Such ET antagonists are set forth in Appendices A through D herein. Additional useful endothelin antagonists can be found in U.S. Patent Application Publication No. US 2002/0082285 A1, incorporated herein by reference.

Specific examples of endothelin antagonists useful in the present invention include, but are not limited to, atrasentan, tezosentan, bosentan, sitaxsentan, enrasentan, BMS-207940 (BristolMyers Squibb), BMS-193884, BMS-182874, J-104132 (Banyu Pharmaceutical), VML 588/Ro 61-1790 (Vanguard Medica), T-0115 (Tanabe Seiyaku), TAK-044 (Takeda), BQ-788, BQ-123, YM-598, LU 135252, PD 145065, A-127722, ABT-627, A-192621, A-182086, TBC3711, BSF208075, S-0139, TBC2576, TBC3214, PD156707, PD180988, ABT-546, ABT-627, Z1611, RPR118031A, SB247083, SB217242, S-Lu302872, TPC10950, and SB209670.

BQ123 is a specific endothelin A antagonist, and is the sodium salt of cyclo(-D-Trp-D-AspPro-D-Val-Leu-). BQ-788 is a specific endothelin B antagonist, and is the sodium salt of N-cis-2,6-dimethylpiperidinocarbonyl-L-gamma-methylleucyl-D-1-methoxycarbonyl triptophanyl-DNIe (see Proc. Natl. Acad. Sci. USA, 91, pp. 4892-4896 (1994)).

In addition to a conventional endothelin antagonist, a compound that inhibits the formation of endogenous endothelin also can be used as the endothelin antagonist in the present invention. Such compounds are useful because they prevent endothelin formation and therefore decrease the activity of endothelin receptors. One class of such compounds is the endothelin converting enzyme (ECE) inhibitors.

Useful ECE inhibitors include, but are not limited to, CGS34225 (i.e., N-((1-((2(S)-(acetylthio)-1-oxopentyl)-amino)-1-cyclopentyl)-carbonyl-S-4-phenylphenyl-alanine methyl ester) and phosphoramidon (i.e., N-(a-rhamnopyranosyloxyhydroxyphosphinyl)-Leu-Trp).

It has been found that an endothelin (ET) receptor antagonist, like BQ123, or an ECE inhibitor can treat AD or a dementia of vascular origin. A vascular theory for dementia of vascular origin and AD has been proposed. Tests and data herein show that endothelin is involved in the action of β-amyloid and, therefore, endothelin antagonists are demonstrated as useful agents to prevent and reduce the progression of dementias of vascular origin and AD.

As previously stated, the differential diagnosis between AD and vascular dementia remains clinically challenging, and the most prominent feature of AD is the extracellular neuritic plaques having β-amyloid (Aβ). Preliminary studies show that Aβ produces significant changes in systemic hemodynamics and regional blood circulation. It also has been shown that Aβ is able to enhance the vasoactivity induced by endothelin-1 (ET-1), and ET-1 expression is increased in the hippocampus and brain stem following treatment with Aβ.

Studies also suggest that ET-1 is an important neuromodulator in the central nervous system (CNS). ET-1, therefore, can act as a neuromodulator by causing a severe reduction of cerebral blood flow. In accordance with the present invention, the vasoactive actions of Aβ mediated through ET are examined. Specifically, the present invention is directed to (1) illustrating the effect of Aβ on systemic hemodynamics and regional blood circulation in rats using a radioactive microsphere technique, and (2) illustrating the effect of Aβ on expression of ET-1 by quantitative RT-PCR.

In particular, rats are treated with vehicle or Aβ (20 μg in three equally divided doses) in the lateral cerebral ventricles using implanted cannula. Drugs are administered at 1, 7, and 14 days, and all experiments are performed on day 15. These experiments are the first to examine the in vivo effect of Aβ on the cardiovascular system and to illustrate that these effects are mediated by ET. Studies using ET antagonists to determine the role of ET in age-related disorders, like AD, also are conducted.

Preliminary studies show that Aβ produces significant changes in systemic hemodynamics and regional blood circulation (FIGS. 2, 3, and 4). ET-1 expression in saline and Aβ-treated rats was determined, and it was found that Aβ increases the ET-1 expression in the hippocampus and brain stem (FIG. 5).

The present invention is of important clinical significance because several ET antagonists are approaching regulatory approval for marketing. On the basis of results obtained in the present studies, it is shown that ET plays an important role in AD, and that ET antagonists are useful in the management of AD and vascular dementias.

### Experimental Design and Methods

The following experiments are performed to illustrate that the vasoactive actions of Aβ are mediated through ET. It was found that Aβ significantly affects the cardiovascular system and increases vascular resistance. It also was found that Aβ significantly increased the ET-1 mRNA expression in the hippocampus and brain stem. Preliminary studies strongly suggest that an interaction exists between Aβ (present in high concentration in AD) and ET (a vasoconstrictor). The following experiments are conducted to determine the effect of Aβ on:

1. systemic hemodynamics and regional blood circulation in rats using a radioactive microsphere technique, and

2. mRNA expression of ET-1 in rats.

### The Effect of Aβ on Systemic Hemodynamics and Regional Blood Circulation in Normotensive and Hypertensive Rats

Rats were anesthetized with ketamine (40 mg/kg, i.m.) and xylazine (4 mg/kg, i.m.) and a lateral cerebral ventricle was cannulated by placing the rat in a stereotaxic (Kopf) instrument and fixing the cannula (using the coordinates: 1.0 mm lateral, 1.5 mm caudal to bregma, and 4.0 mm deep from the bone) with dental cement. The animals were allowed to recover from surgery for at least seven days. Volume of drug injection was 5 μl over a period of 5 minutes, and after each experiment, methylene blue dye (5 μl) was injected and the placement of the cannula was confirmed by observing the site and extent of staining. After seven days, rats were treated with vehicle or Aβ (20 μg in 3 equally divided doses) in the lateral cerebral ventricles using implanted cannula. Drugs were administered on days 1, 7, and 14, and all the experiments were performed on day 15.

Male Fischer 344 rats at 4, 15, and 24 months of age were anesthetized with urethane (1.2 g/kg, ip). The carotid artery of the right side was exposed, and a PE 50 cannula guided through the common carotid artery to the left ventricle. The presence of the cannula in the left ventricle was confirmed by recording the pressure on the Grass polygraph using Statham P 23 DC pressure transducer. When the cannula reaches into the ventricle, diastolic pressure drops to zero.

At each measurement, a suspension of approximately 100,000 microspheres (15±1 μm diameters) labeled with ⁴⁶Sc (scandium), ¹¹³Sn (tin), ¹⁴¹Ce (cerium), or ¹⁰³Ru (ruthenium) (New England Nuclear Corporation, Boston, Mass.) in 0.2 ml saline were injected into the left ventricle after thoroughly mixing and flushed with 0.4 ml saline over 15 second period. To calculate blood flow, arterial blood was withdrawn at a rate of 0.5 ml/min through the catheter inserted in the abdominal aorta via the right femoral artery for 90 seconds starting about 5 to 10 seconds before the microsphere injection. The animals were sacrificed following the experiment with an overdose of pentobarbital sodium, and the brain regions (cerebral hemisphere, midbrain, hypothalamus, pituitary, cerebellum, pons, and medulla) are dissected out, weighed, and placed in vials containing 10% formalin. The radioactivity of the microspheres injected, the blood samples, and the brain samples were determined using a Packard Minaxi AutoGamma 5000 series gamma counter with preset windows discriminating the isotope energies. Systemic hemodynamics, and regional blood flow and vascular resistance, were calculated using a software program (Saxena et al., 1980).

Experiments in rats were performed to determine systemic hemodynamics and regional blood circulation in control (saline) and Aβ-treated rats.

mRNA studies performed in rats show that Aβ produces significant increase in expression of ET-1 in aged rats. Although, mRNA studies have shown that the CNS contains substantial ET_{B} receptor mRNA, while ET_{A} receptor mRNA levels are extremely low, pharmacological studies demonstrate that most of the central ET responses are mediated through ET_{A} receptor and not ET_{B} receptors.

Studies have shown that although Aβ produces severe cerebral vasoconstriction, the effect of ET-1 infusion on brain blood flow is more marked in Aβ-treated rats. It was found that during ET-1 infusion (30 minutes), cerebral vasoconstriction was produced, which was more marked in Aβ-treated rats. Once infusion was stopped, a rebound increase in blood flow was observed. It can be inferred that Aβ increased the endogenous concentration of ET-1, leading to vasoconstriction. Accordingly, the administration of an ET-1 antagonist can cause a rebound increase in blood flow.

The Effect of Aβ on mRNA Expression of ET-1

Brain regions were taken from rats on day 15, treated with either saline or Aβ, as described above, to determine the mRNA expression of ET-1. These techniques have been performed extensively in the past (Evans et al., 1996; Gulati et al., 1998; Gulati et al., 1992: Iyer et al., 1995; Murlas et al., 1997). mRNA expression studies by RT-PCR also have been standardized. An example of ET-1 expression in saline and β-amyloid-treated rats is shown in FIG. 5. Briefly, the following procedure is followed:

Isolation of total RNA: Qiagen RNeasy kit (Qiagen) is used to isolate total RNA. The frozen segments of brain (cerebellum, cerebral cortex, brain stem, hypothalamus, hippocampus, pituitary, diencephalons), approximately 30 mg each, were minced under liquid nitrogen with the aid of an electric grinder. RNA is extracted as per manufacturer's instructions. Quantitation of RNA is performed by determining the absorbance at 260 and 280 nm.

First-strand cDNA synthesis: First-strand cDNA synthesis is performed using a Superscript-II system (GIBCO, Life Technologies) in a total volume of 30 μL according to manufacturer's instructions. Reverse transcription is performed in a 30-μL volume containing 1-3 μg RNA, 1.5 μL of 10 mmol/L dNTP, 6 μL of BRL 5×buffer, 0.6 μL of oligo-(dt)12-18 primer (0.5 μg/μL), 1.5 μL of 200 U/μL M-MLV reverse transcriptase (BIGCO-BRL), 0.9 μL of rRNasin (RNase inhibitor; 40 U/μL), and 3 μL of dithiothreitol (0.1 mol/L) for 1 hour at 42° C. The reaction is stopped by heating at 70° C. for 15 minutes.

Relative RT-PCR Analysis of PreproET-1 mRNA: 1 μL of the resulting cDNA solution and 1.0 μmol/L (GIBCO) of each primer were used for PCR with 1 U Pfx DNA polymerase (GIBCO) in a total volume of 50 μL according to the manufacturer's instructions. PCR conditions described for ET-1 were identical for the other gene products except for the individual adjustment of cDNA volumes. All PCR reactions were performed individually for each primer pair in a Techne Cyclogene thermocycler that is programmed as follows: a unique 3-minute period for complete denaturation at 94° C. in the beginning followed by a primer-specific number of cycles of 30 second denaturation at 94° C., 30-second annealing at 50° C. to 60° C. (see below), and 1-minute primer extension at 72° C., with an additional 7 minutes at 72° C. for final extension in the end. Simultaneously, a control gene (GAPDH) is amplified in a separate set of tubes using the RT product (primer set: 5′CAACTTGATCCACGTTCACC3′ and 5′GAAGAGCCAAGGACAGGTAC3′) using similar cycling parameters with a predicted product size of 270 bp. RT-PCR products were electrophoretically separated on 1.5% agarose gels containing 0.1% ethidium bromide, and the intensity of the detected bands are determined. Band intensity is quantified using Gel Doc 1000 darkroom imager and molecular analyst software (Bio-Rad Laboratories, Hercules, Calif.). The amount of DNA in each specimen is quantified by integrated density of the product bands within a close rectangle, which then is normalized to the volume of GAPDH bands.

Oligonucleotide Primers for PreproET-1, PreproET-3, the ETA Receptor, the ETB Receptor, and GAPDH used for PCR: PCR primers have been designed on the basis of published rat cDNA sequences for preproET-1, preproET-3, ET_{A} receptor, ET_{B} receptor.

Prepro-ET-1: product size 319 by 30 cycles, 50° C.; sense 5′CTAGGTCTAAGCGACCTTG3′; anti-sence 5′TCTGGTCTCTGTAGAGTTC3′

ET_{A}-R: product size 188 bp, 30 cycles, 55° C.; sense 5′CCTTATCTACGTGGTCATTGATCT3′; antisense 5′AAGCCACTGCTCTGTACCTG3′

ET_{B}-R: product size 304 bp, 30 cycles, 55° C.; sense 5′TGTTGGCTTCCCCTTCATCT3′; antisense 5′TGGAGCGGAAGTTGTCGTAT3′

GAPDH: product size 254 bp, 30 cycles, 55° C.; sense 5′TATGATGACATCAAGAAGGTGG3′; antisense 3′ATGTCGTTGTCCCACCAC-5′

ECE-1: product size 529 bp, 30 cycles, 54° C.; sense 5′CGTAGCGATAGTCTTAGCAC3′; antisense 5′GTGCCACACCAAAACTACAG3′.

To verify the identify of the amplification products with the designed primer pairs, the ET-1, ETB-R, GAPDH PCR products were sequenced, and an 87% to 92% homology with the published sequences of the corresponding rat genes was found.

The endothelin antagonist can be formulated in suitable excipients for oral administration or for parenteral administration. Such excipients are well known in the art. The endothelin antagonists typically are present in such a composition in an amount of about 0.1% to about 75% by weight.

Pharmaceutical compositions containing the endothelin antagonist are suitable for administration to humans or other mammals. Typically, the pharmaceutical compositions are sterile, and contain no toxic, carcinogenic, or mutagenic compounds that would cause an adverse reaction when administered.

The method of the present invention can be accomplished using an endothelin antagonist as described above, or as a physiologically acceptable salt, derivative, prodrug, or solvate thereof. The endothelin antagonist, or a form thereof, can be administered as the neat compound, or as a pharmaceutical composition. Administration of the pharmaceutical composition, or individual endothelin antagonists, can be performed before, during, or after the onset of AD or dementia of vascular origin.

The endothelin antagonists also can be administered in conjunction with a second therapeutic agent useful in the treatment of AD or a dementia of vascular origin. The second therapeutic agent is different from an endothelin antagonist. The endothelin antagonist and second therapeutic agent can be administered simultaneously or sequentially. In addition, the endothelin antagonist and second therapeutic agent can be administered from a single composition or two separate compositions.

Nonlimiting examples of second therapeutic agents include, but are not limited to, tacrine (i.e., tetrahydroaminoacridine), metrifonate, bethanacol, physostigmine, donepezil, rivastigmine, galantamine, and other cholinesterase inhibitors. Another class of second therapeutic agents is the statins, including, but not limited to atorvastatin, fluvastatin, lovastatin, and pravastatin. Additional second therapeutic agents include, but are not limited to, memantine, CX516 (e.g., AMPALEX, an ampakine from Cortex Pharmaceuticals, Inc.), AN-1792 (a form of β-amyloid 42), and nonsteroidal antiinflammatory drugs, like aspirin and acetaminophen. Also envisioned as second therapeutic agents are devices used to treat Alzheimer's disease, such as COGNIShunt.

The endothelin antagonists can be administered by any suitable route, for example by oral, buccal, inhalation, sublingual, rectal, vaginal, intracisternal or intrathecal through lumbar puncture, transurethral, nasal, percutaneous, i.e., transdermal, or parenteral (including intravenous, intramuscular, subcutaneous, and intracoronary) administration. Parenteral administration can be accomplished using a needle and syringe, or using a high pressure technique, like POWDERJECT™.

The pharmaceutical compositions include those wherein the endothelin antagonists are administered in an effective amount to achieve their intended purpose. More specifically, a “therapeutically effective amount” means an amount effective to ameliorate, eliminate, or retard the progression of Alzheimer's disease or a dementia of vascular origin. Determination of a therapeutically effective amount is well within the capability of those skilled in the art, especially in light of the detailed disclosure provided herein.

A “therapeutically effective dose” refers to that amount of the endothelin antagonists that results in achieving the desired effect. Toxicity and therapeutic efficacy of such endothelin antagonists can be determined by standard pharmaceutical procedures in cell cultures or experimental animals, e.g., determining the LD₅₀ (the dose lethal to 50% of the population) and the ED₅₀ (the dose therapeutically effective in 50% of the population). The dose ratio between toxic and therapeutic effects is the therapeutic index, which is expressed as the ratio between LD₅₀ and ED₅₀. A high therapeutic index is preferred. The data obtained can be used in formulating a range of dosage for use in humans. The dosage of the active agents preferably lies within a range of circulating concentrations that include the ED₅₀ with little or no toxicity. The dosage can vary within this range depending upon the dosage form employed, and the route of administration utilized.

The exact formulation, route of administration, and dosage is determined by an individual physician in view of the patient's condition. Dosage amount and interval can be adjusted individually to provide levels of the endothelin antagonists that are sufficient to maintain therapeutic or prophylactic effects.

The amount of pharmaceutical composition administered is dependent on the subject being treated, on the subject's weight, the severity of the affliction, the manner of administration, and the judgment of the prescribing physician.

Specifically, for administration to a human in the curative or prophylactic treatment of AD or a vascular dementia, oral dosages of an endothelin antagonist, individually generally are about 10 to about 200 mg daily for an average adult patient (70 kg), typically divided into two to three doses per day. Thus, for a typical adult patient, individual tablets or capsules contain about 0.1 to about 50 mg endothelin antagonist, in a suitable pharmaceutically acceptable vehicle or carrier, for administration in single or multiple doses, once or several times per day. Dosages for intravenous, buccal, or sublingual administration typically are about 0.1 to about 10 mg/kg per single dose as required. In practice, the physician determines the actual dosing regimen that is most suitable for an individual patient, and the dosage varies with the age, weight, and response of the particular patient. The above dosages are exemplary of the average case, but there can be individual instances in which higher or lower dosages are merited, and such are within the scope of this invention.

The endothelin antagonists can be administered alone, or in admixture with a pharmaceutical carrier selected with regard to the intended route of administration and standard pharmaceutical practice. Pharmaceutical compositions for use in accordance with the present invention thus can be formulated in a conventional manner using one or more physiologically acceptable carriers comprising excipients and auxiliaries that facilitate processing of the endothelin antagonists into preparations which can be used pharmaceutically.

These pharmaceutical compositions can be manufactured in a conventional manner, e.g., by conventional mixing, dissolving, granulating, dragee-making, emulsifying, encapsulating, entrapping, or lyophilizing processes. Proper formulation is dependent upon the route of administration chosen. When a therapeutically effective amount of the endothelin antagonists are administered orally, the composition typically is in the form of a tablet, capsule, powder, solution, or elixir. When administered in tablet form, the composition additionally can contain a solid carrier, such as a gelatin or an adjuvant. The tablet, capsule, and powder contain about 5% to about 95% of an endothelin antagonist, and preferably from about 25% to about 90% endothelin antagonist. When administered in liquid form, a liquid carrier, such as water, petroleum, or oils of animal or plant origin, can be added. The liquid form of the composition can further contain physiological saline solution, dextrose or other saccharide solutions, or glycols. When administered in liquid form, the composition contains about 0.5% to about 90% by weight of endothelin antagonists, and preferably about 1% to about 50% of endothelin antagonists.

When a therapeutically effective amount of the endothelin antagonist is administered by intravenous, cutaneous, or subcutaneous injection, the composition is in the form of a pyrogen-free, parenterally acceptable aqueous solution. The preparation of such parenterally acceptable solutions, having due regard to pH, isotonicity, stability, and the like, is within the skill in the art. A preferred composition for intravenous, cutaneous, or subcutaneous injection typically contains, in addition to an isotonic vehicle.

Suitable endothelin antagonists can be readily combined with pharmaceutically acceptable carriers well-known in the art. Such carriers enable the active agents to be formulated as tablets, pills, dragees, capsules, liquids, gels, syrups, slurries, suspensions and the like, for oral ingestion by a patient to be treated. Pharmaceutical preparations for oral use can be obtained by adding the endothelin antagonists with a solid excipient, optionally grinding the resulting mixture, and processing the mixture of granules, after adding suitable auxiliaries, if desired, to obtain tablets or dragee cores. Suitable excipients include, for example, fillers and cellulose preparations. If desired, disintegrating agents can be added.

The endothelin antagonists can be formulated for parenteral administration by injection, e.g., by bolus injection or continuous infusion. Formulations for injection can be presented in unit dosage form, e.g., in ampules or in multidose containers, with an added preservative. The compositions can take such forms as suspensions, solutions, or emulsions in oily or aqueous vehicles, and can contain formulatory agents such as suspending, stabilizing, and/or dispersing agents.

Pharmaceutical compositions for parenteral administration include aqueous solutions of the active agent in water-soluble form. Additionally, suspensions of the endothelin antagonists can be prepared as appropriate oily injection suspensions. Suitable lipophilic solvents or vehicles include fatty oils or synthetic fatty acid esters. Aqueous injection suspensions can contain substances which increase the viscosity of the suspension. Optionally, the suspension also can contain suitable stabilizers or agents that increase the solubility of the compounds and allow for the preparation of highly concentrated solutions. Alternatively, a present composition can be in powder form for constitution with a suitable vehicle, e.g., sterile pyrogen-free water, before use.

The endothelin antagonists also can be formulated in rectal compositions, such as suppositories or retention enemas, e.g., containing conventional suppository bases. In addition to the formulations described previously, the endothelin antagonists also can be formulated as a depot preparation. Such long-acting formulations can be administered by implantation (for example, subcutaneously or intramuscularly) or by intramuscular injection. Thus, for example, the endothelin antagonists can be formulated with suitable polymeric or hydrophobic materials (for example, as an emulsion in an acceptable oil) or ion exchange resins, or as sparingly soluble derivatives, for example, as a sparingly soluble salt.

In particular, the endothelin antagonists can be administered orally, buccally, or sublingually in the form of tablets containing excipients, such as starch or lactose, or in capsules or ovules, either alone or in admixture with excipients, or in the form of elixirs or suspensions containing flavoring or coloring agents. Such liquid preparations can be prepared with pharmaceutically acceptable additives, such as suspending agents. The endothelin antagonists also can be injected parenterally, for example, intravenously, intramuscularly, subcutaneously, or intracoronarily. For parenteral administration, the endothelin antagonists are best used in the form of a sterile aqueous solution which can contain other substances, for example, salts, or monosaccharides, such as mannitol or glucose, to make the solution isotonic with blood.

For veterinary use, the endothelin antagonists are administered as a suitably acceptable formulation in accordance with normal veterinary practice. The veterinarian can readily determine the dosing regimen and route of administration that is most appropriate for a particular animal.

### REFERENCES

O. Almkvist et al., Eur. Arch. Psychiatry Clin. Neurosci, 249, (Suppl 3), 3-9, 1999.
H. Arai et al., Nature, 348, (6303), 730-2, 1990.
M. Barton et al., J. Cardiovasc Pharmacol, 36, (5 Suppl 1), S153-6, 2000.
G. A. Bertelsen et al., Neurobiol Aging, 13(4), 513-9, 1992.
H. J. Cohen, Cancer, 80(7), 1294-1301, 1997.
P. D. Coleman et al., Neurobiol Aging, 8(6), 521-45, 1987(a).
P. D. Coleman et al., J Comp Neurol, 266(2), 300-6, 1987(b).
P. O. Couraud et al., Life Sci, 49(20), 1471-6, 1991.
F. Crawford et al., FEBS Lett, 436(3), 445-8, 1998(a).
F. Crawford et al., Exp Neurol, 150(1), 159-68, 1998(b).
H. Ehrenreich et al., Brain Res, 538 (1), 54-8, 1991.
R. R. Evans et al., Am J. Cardiol, 78(4), 486-8, 1996.
W. Goettsch et al., Biochem Biophys Res Commun, 280(3), 908-13, 2001.
G. Gold et al., Ann Med Interne (Paris), 149(4), 202-8, 1998.
D. A. Greenberg et al., Neurology, 42(1), 25-31, 1992.
A. Gulati et al., Am J. Hypertens, 11(7), 866-9, 1998.
A. Gulati et al., Life Sci, 58(5), 437-45, 1996.
A. Gulati et al., Life Sci., 48(12), 1207-15, 1991.
A. Gulati et al., Am J. Physiol, 273(3 Pt 2), H1177-86, 1997.
A. Gulati et al., J. Cardiovasc Pharmacol, 26(Suppl 3), S244-6, 1995.
A. Gulati et al., Drug Develop Res, 26, 361-387, 1992.
S. P. Harland et al., J. Cardiovasc Pharmacol, 26 (Suppl 3), S408-11, 1995.
J. Hensen et al., Z. Kardiol, 80(Suppl 8), 101-2, 1991.
E. Hosli et al., Neurosci Lett, 129(1), 55-8, 1991.
M. Iemitsu et al., Clinical Sci. (London), 103(51), 1525-1575 (2002).
R. S. Iyer et al., Pharmacology, 51(2), 96-104, 1995.
W. J. Jugust et al., Annals of Neurology, 44, 258-262, 1987.
M. Kohzuki et al., J. Cardiovasc Pharmacol, 26 (Suppl 3), S329-31, 1995.
S. Koizumi et al., Neuroreport, 5(9), 1077-80, 1994.
A. Kumar et al., Gen Pharmacol, 27(6), 1025-33, 1996.
M. E. Lee et al., J. Clin Invest, 86(1), 141-7, 1990.
T. F. Luscher et al., J. Cardiovasc Pharmacol, 19 (Suppl 5), S34-42, 1992.
M. W. MacCumber et al., Proc Natl Acad Sci USA, 87(6), 2359-63, 1990.
P. Marin et al., J. Neurochem, 56(4), 1270-5, 1991.
J. Maslarova et al., Methods Find Exp Clin Pharmacol, 17(7), 431-6, 1995.
C. G. Murlas et al., Lung, 175(2), 117-26, 1997.
S. Rebello et al., Brain Res. 676(1), 141-50, 1995.
O. Sagher et al., Brain Res, 658(1-2), 179-84, 1994.
T. Sakurai et al., Nature, 348(6303), 732-5, 1990.
P. R. Saxena et al., Comput Programs Biomed, 12(2-3), 63-84, 1980.
J. M. Starr et al., Age Ageing, 22(5), 343-8, 1993.
K. Takahashi et al., J. Cardiovasc Pharmacol, 17 (Suppl 7), S101-3, 1991.
L. D. Thomas, J. Nurs Pract, 2(1), 29-32, 1996.
M. White et al., Clinical Sci. (London), 93(4), 309-316 (1997).
L. C. Wilkes et al., Br J Pharmacol, 104(3), 750-4, 1991.
M. Yanagisawa et al., J Hypertens, Suppl 6(4), S188-91, 1988.
R. Yancik, J. Gerontol A Biol Sci Med Sci, 52(6), M329-32, 1997.
T. Yoshizawa et al., Science, 247(4941), 462-4, 1990.
J. C. Yu ec al., Br J Pharmacol, 116(5), 2441-6, 1995.
W. W. Zhang et al., Dementia, 5(3-4), 153-62, 1994(a).
W. W. Zhang et al., J. Neurol Sci, 122 (1), 90-6, 1994(b).

Modifications and variations of the invention as hereinbefore set forth can be made without departing from the spirit and scope thereof, and, therefore, only such limitations should be imposed as are indicated by the appended claims.

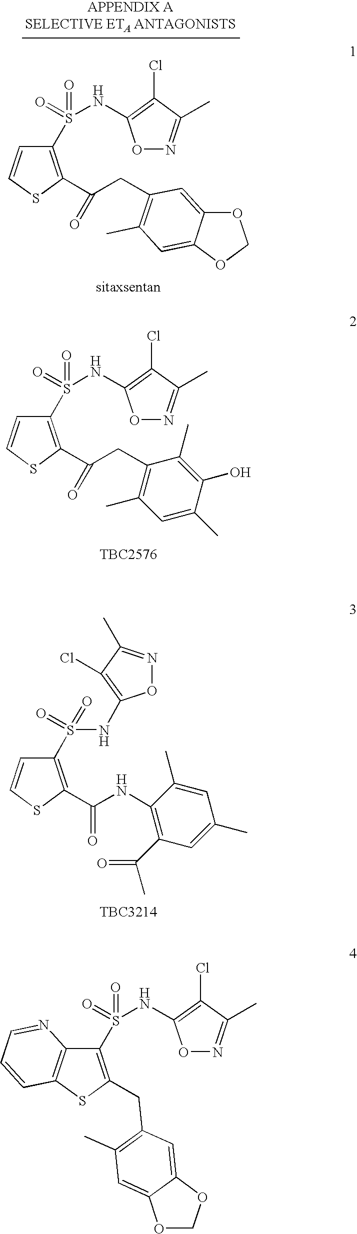

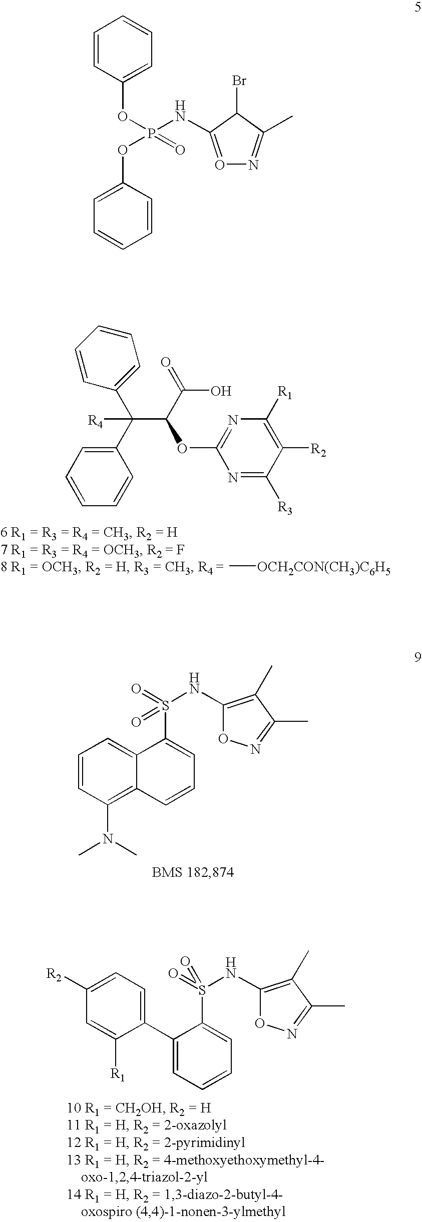

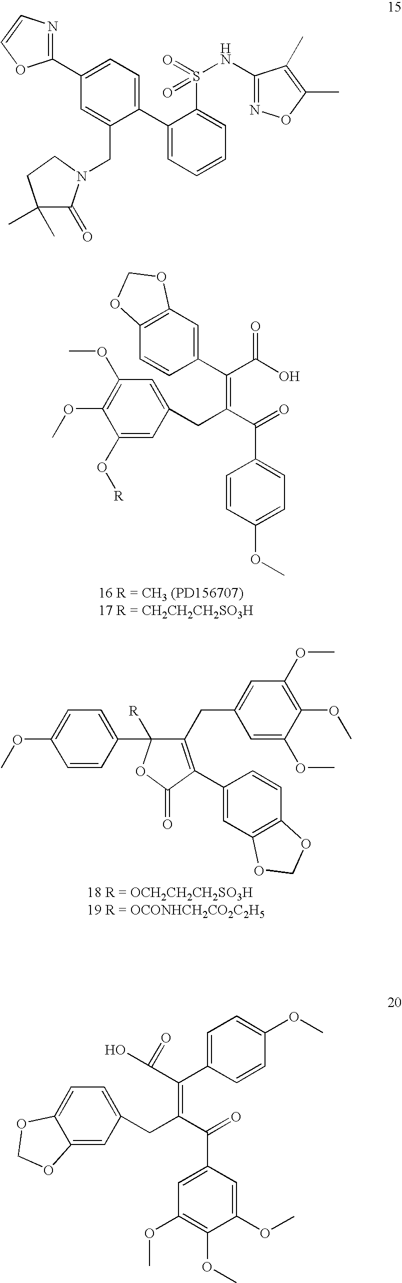

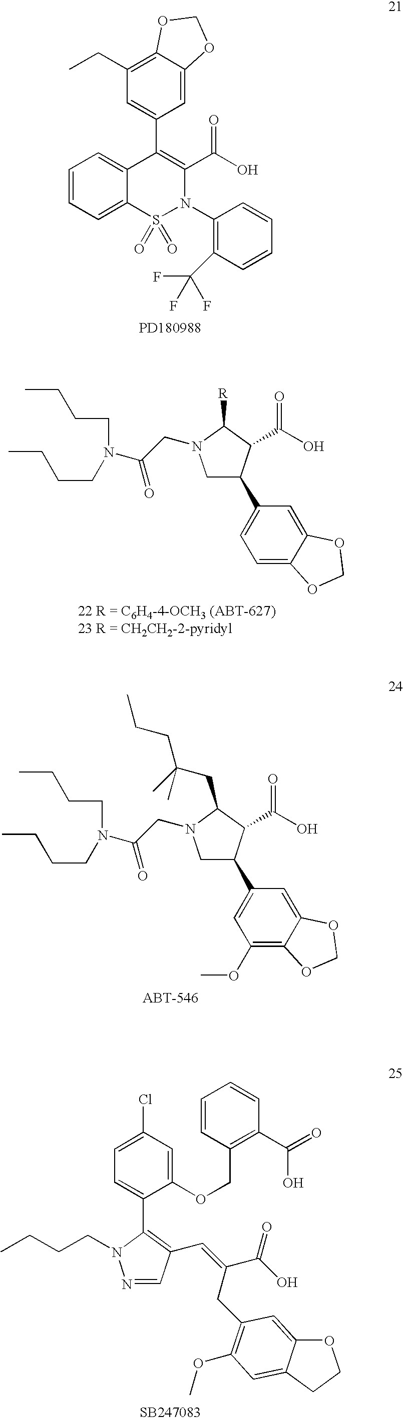

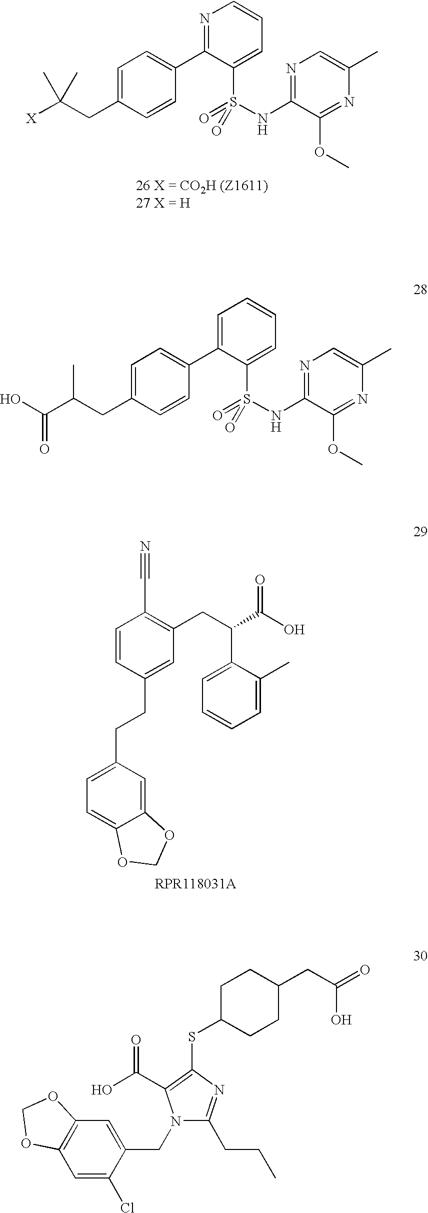

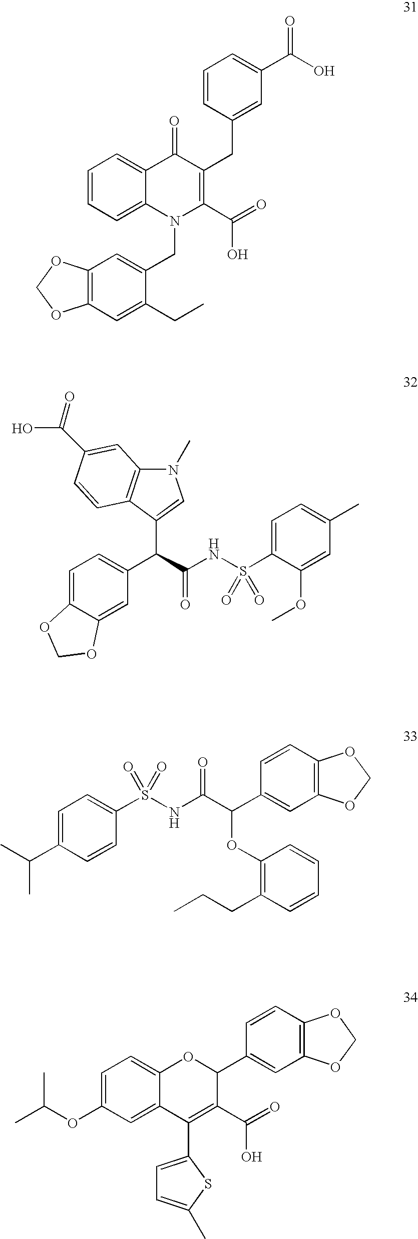

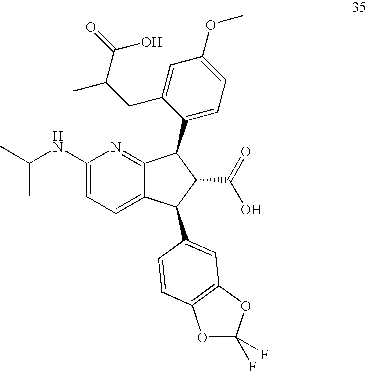

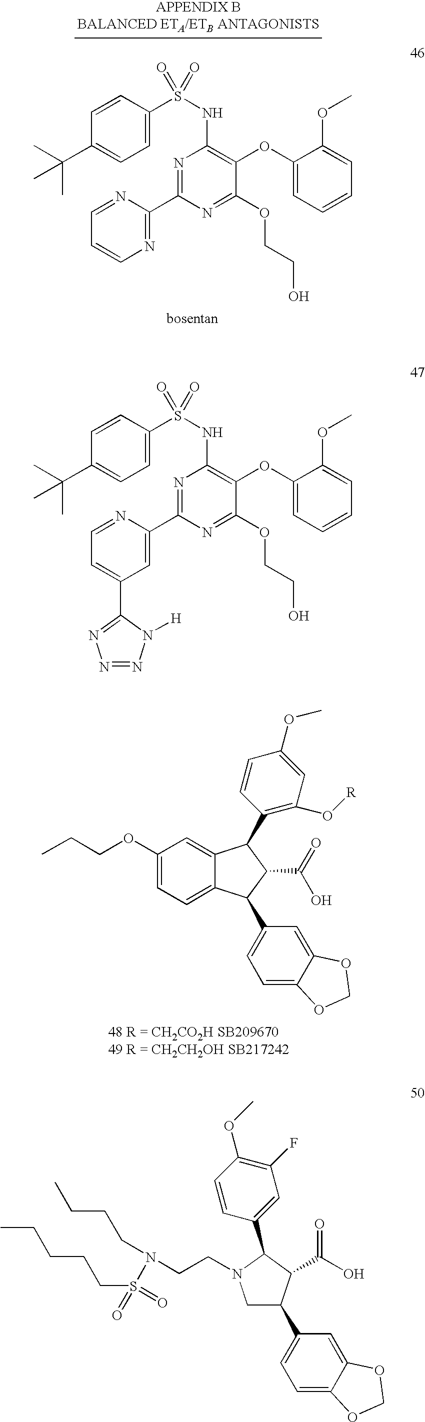

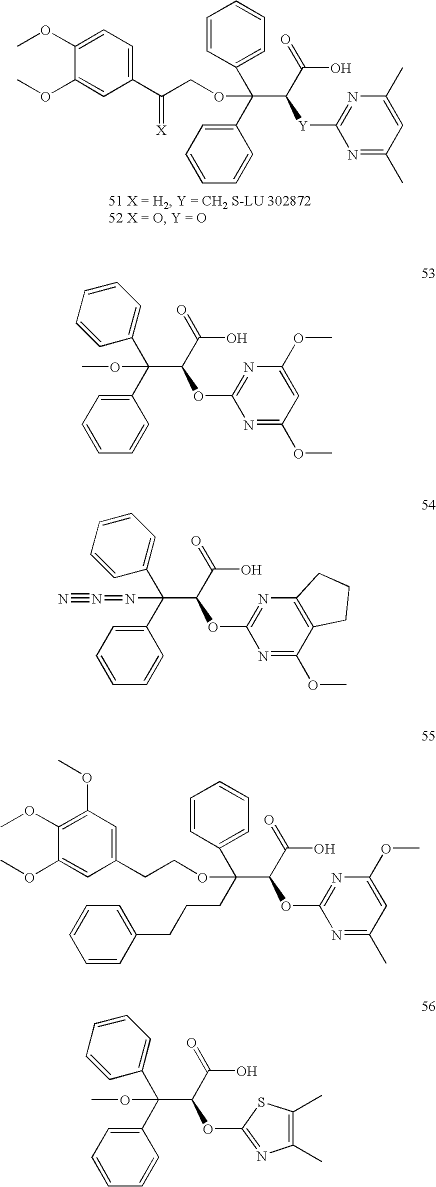

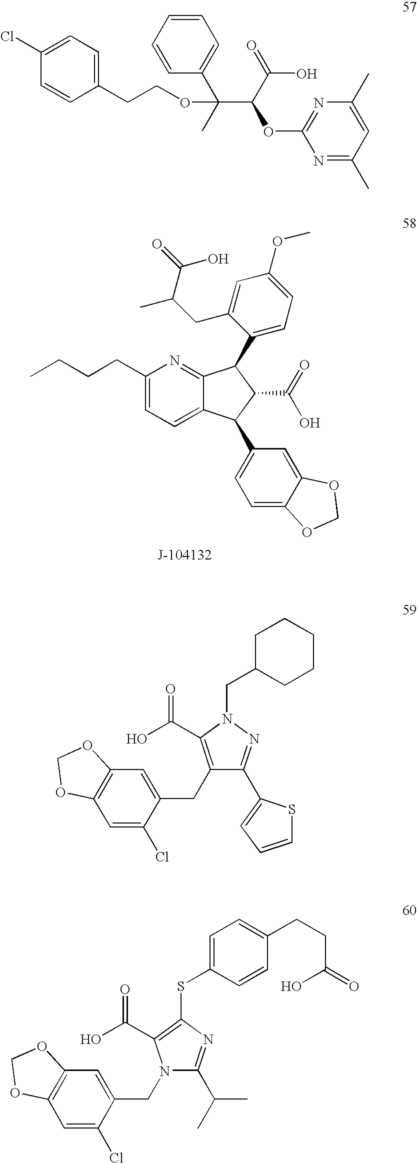

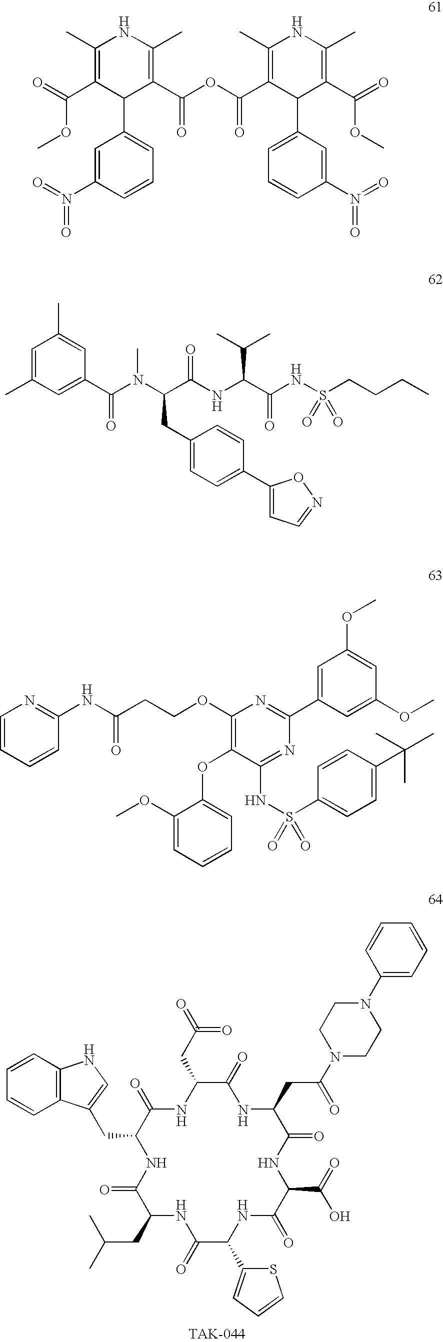

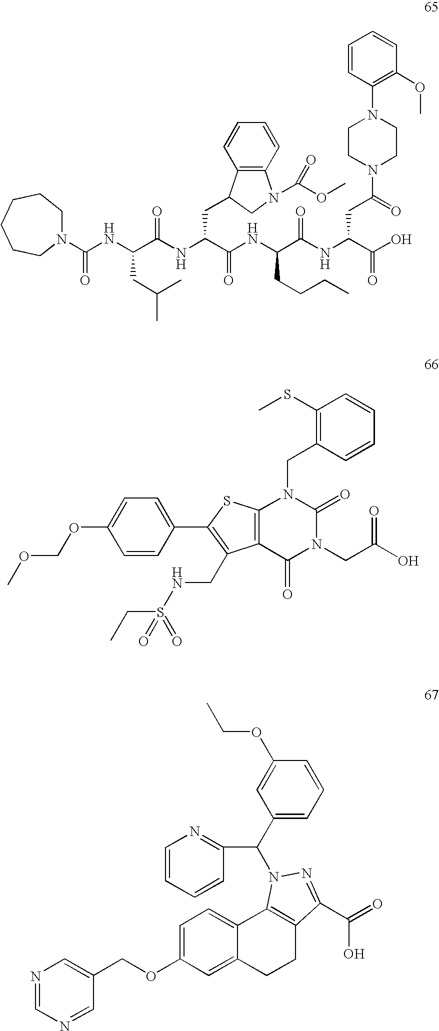

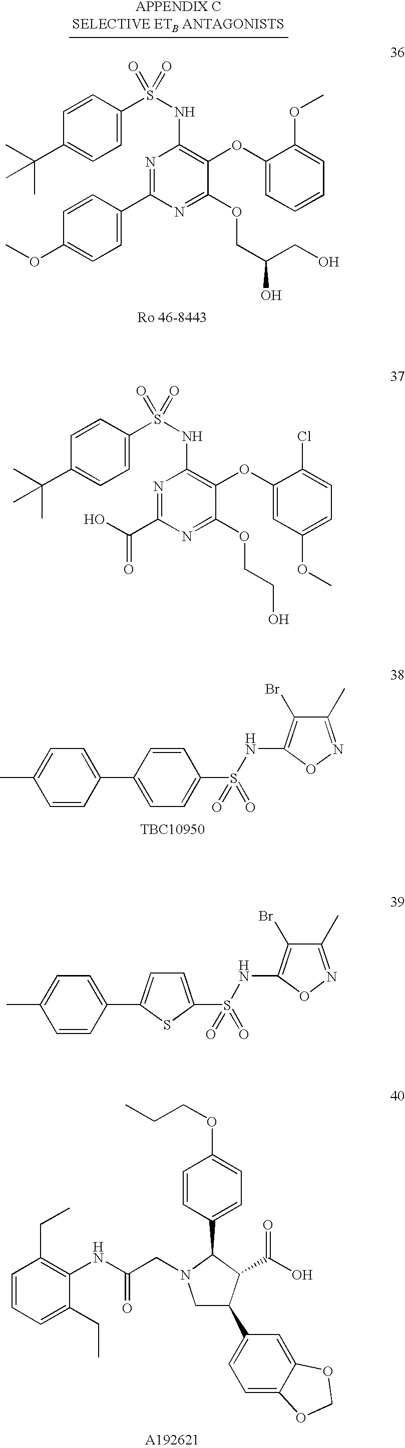

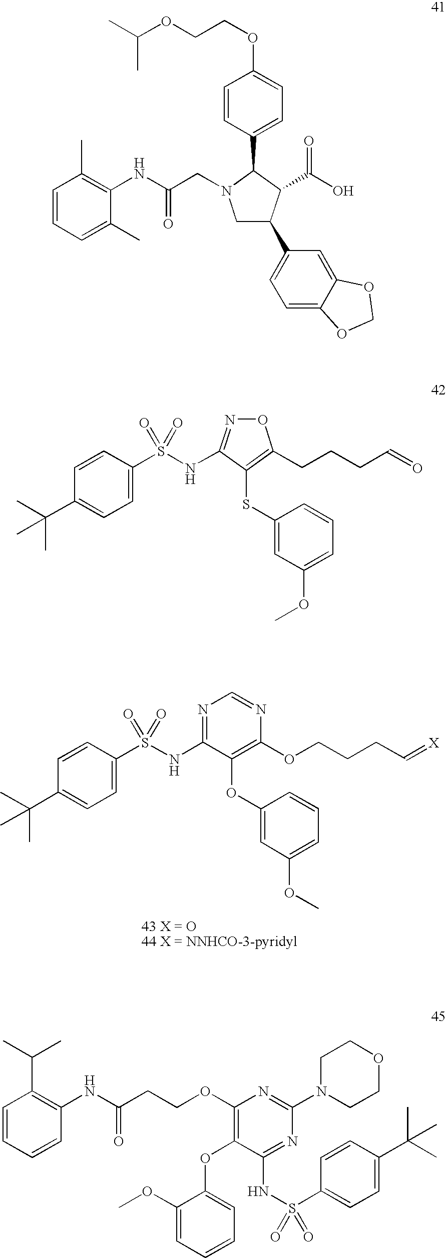

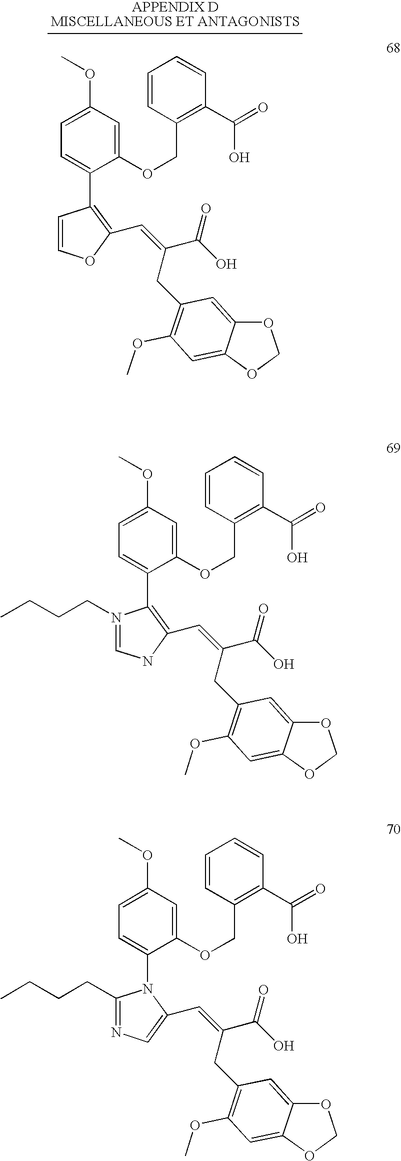

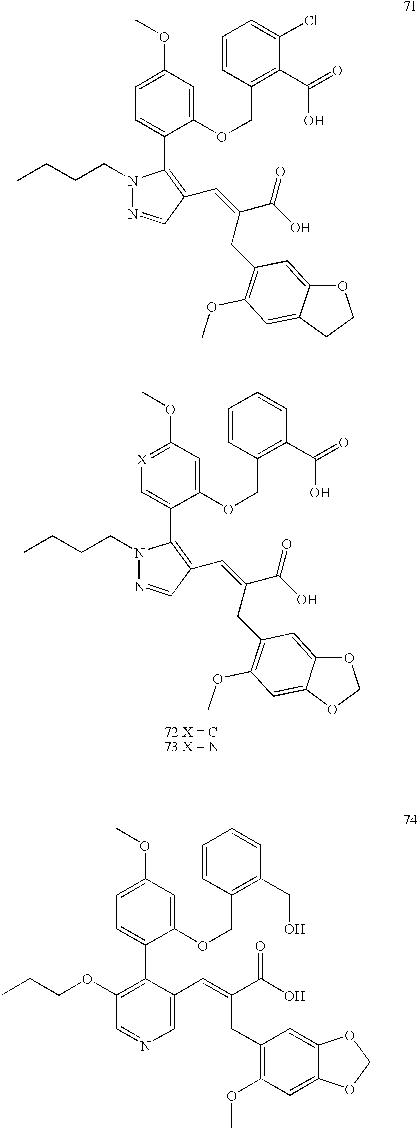

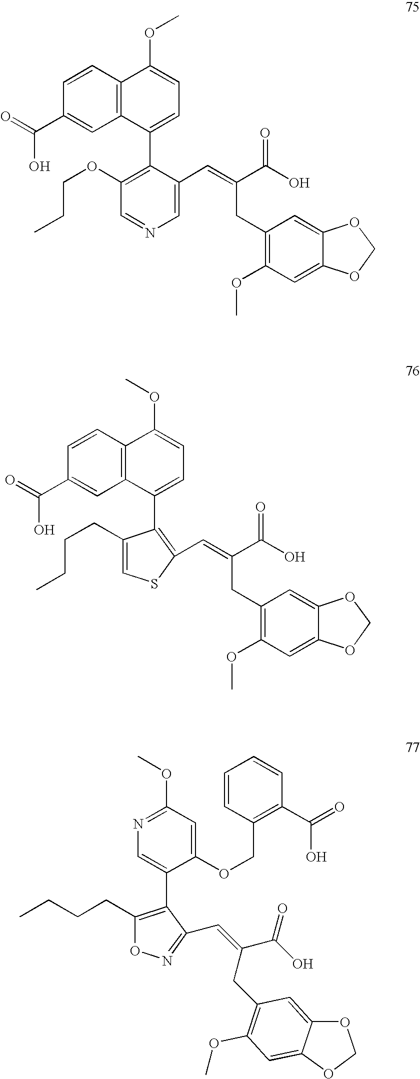

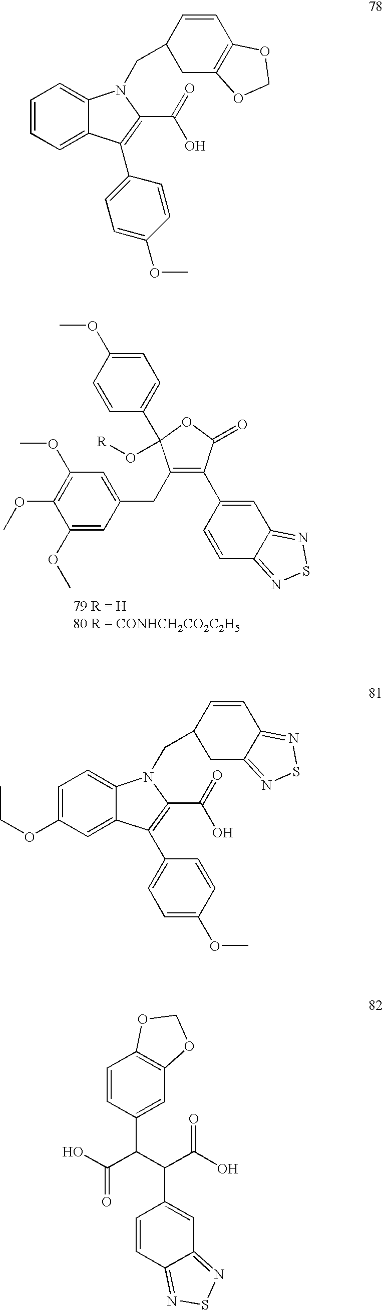

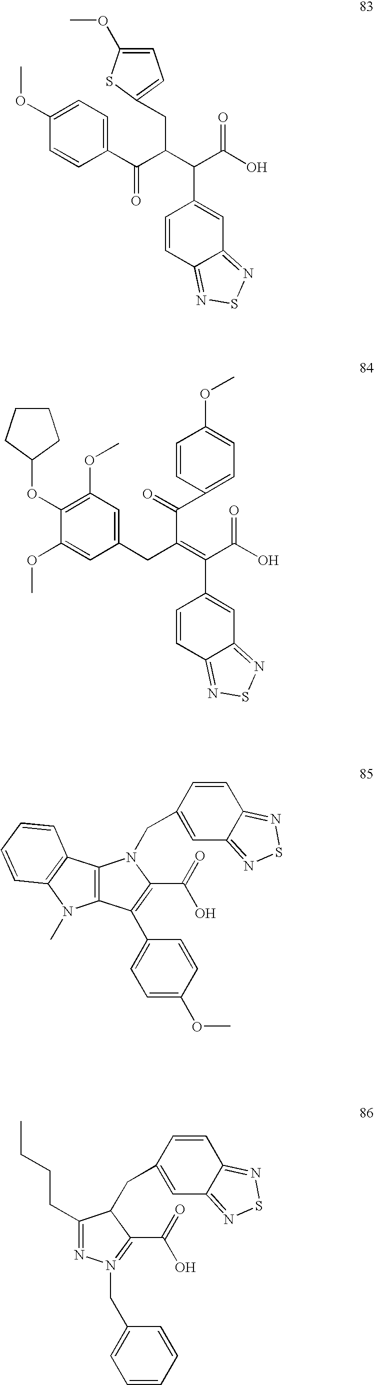

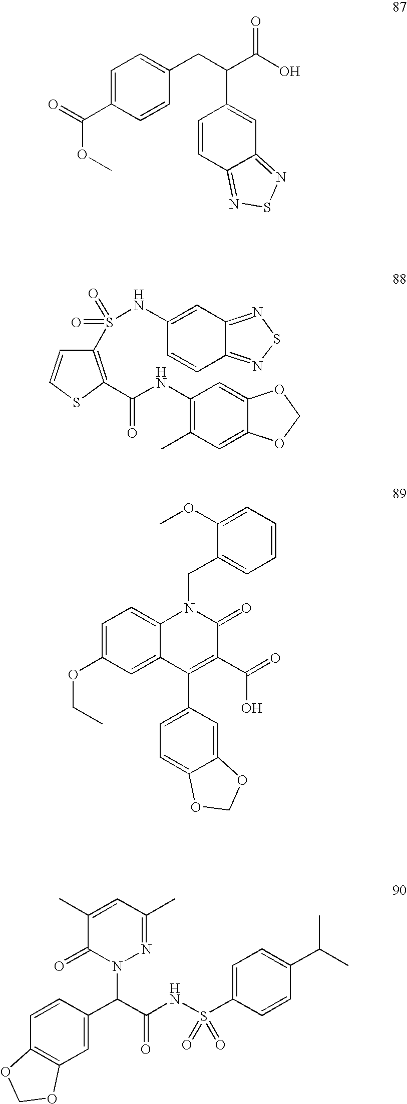

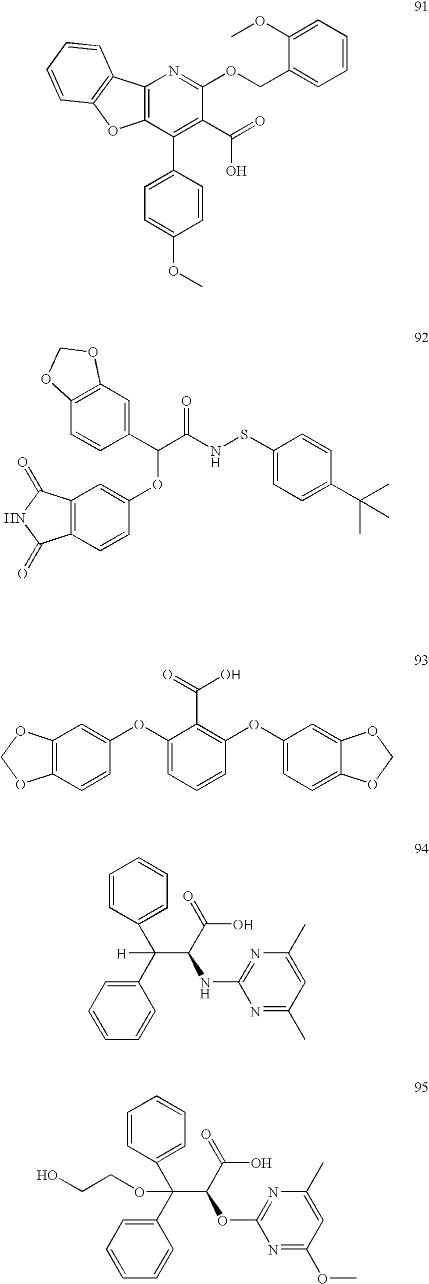

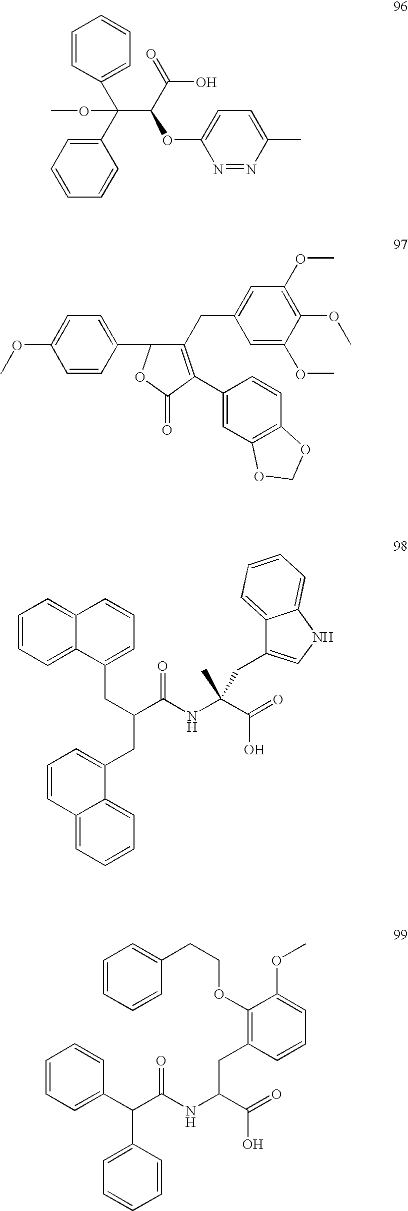

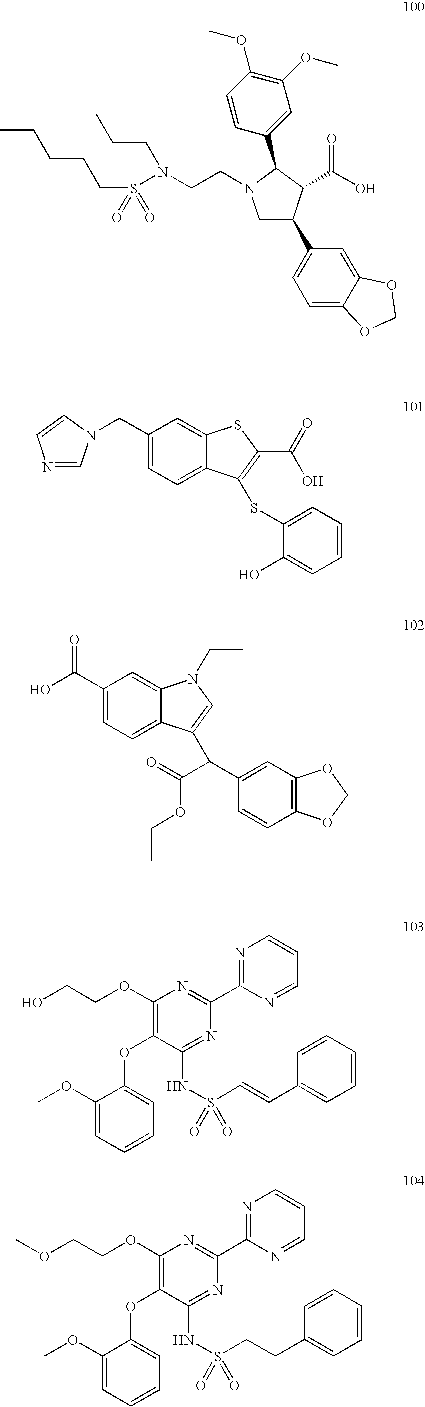

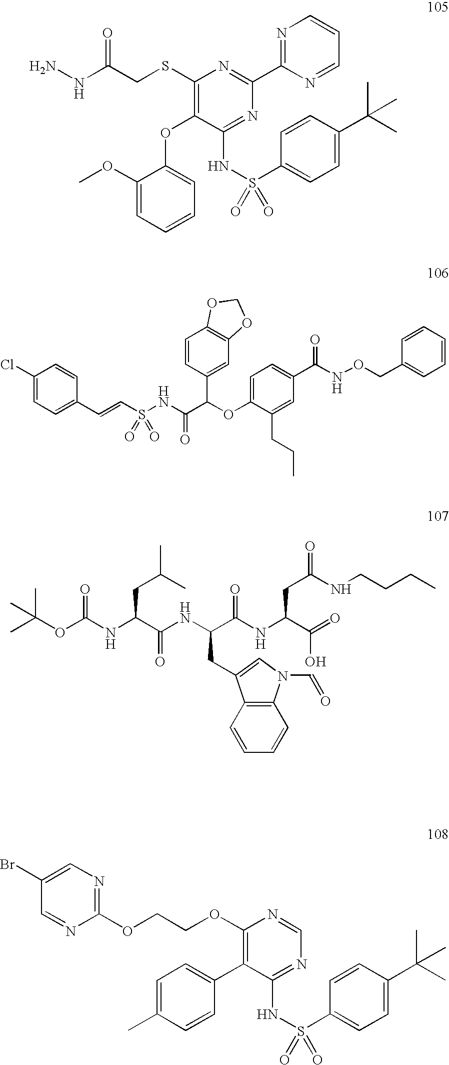

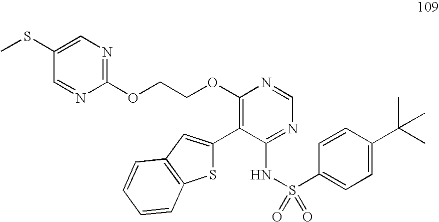

1. A method of treating Alzheimer's disease or a dementia of vascular origin comprising administering to a mammal in need thereof a therapeutically effective amount of a nonspecific endothelin-A antagonist.

2. (canceled)

3. (canceled)

4. (canceled)

5. (canceled)

6. The method of claim 1 wherein the endothelin antagonist is selected from the group consisting of compounds 36 through 45 of Appendix C.

7. (canceled)

8. (canceled)

9. The method of claim 1 wherein the endothelin antagonist is selected from the group consisting of bosentan, sitaxsentan, enrasentan, BMS-207940, BMS-193884, and mixtures thereof.

10. (canceled)

11. (canceled)

12. (canceled)

13. The method of claim 1 further comprising administering a therapeutically effective amount of a second therapeutic agent useful in the treatment of Alzheimer's disease or a dementia of vascular origin.

14. The method of claim 13 wherein the second therapeutic agent comprises a cholinesterase inhibitor.

15. The method of claim 14 wherein the cholinesterase inhibitor is selected from the group consisting of tacrine, metrifonate, bethanacol, physostigmine, donapezil, rivastigmine, galantamine, and mixtures thereof.

16. The method of claim 13 wherein the second therapeutic agent comprises a statin.

17. The method of claim 16 wherein the statin is selected from the group consisting of atorvastatin, fluvastatin, lovastatin, pravastatin, and mixtures thereof.

18. The method of claim 13 wherein the second therapeutic agent is selected from the group consisting of a nonsteroidal antiinflammatory drug, memantine, CX516, AN-1792, COSNIShunt, and mixtures thereof.

19. The method of claim 13 wherein the endothelin antagonist and second therapeutic agent are administered simultaneously.

20. The method of claim 13 wherein the endothelin antagonist and second therapeutic agent are administered from a single composition.

21. The method of claim 13 wherein the endothelin antagonist and second therapeutic agent are administered from separate compositions.

22. The method of claim 13 wherein the endothelin antagonist and second therapeutic agent are administered separately.

23. The method of claim 22 wherein the endothelin antagonist is administered prior to the second therapeutic agent.

24. The method of claim 22 wherein the endothelin antagonist is administered after the second therapeutic agent.

25. The method of claim 1 wherein the mammal is a human.

26. (canceled)

27. (canceled)

28. (canceled)

29. A composition comprising (a) a nonspecific endothelin-A antagonist, (b) a second therapeutic agent useful in the treatment of Alzheimer's disease or a dementia of vascular origin, and (c) an optional excipient.

30. (canceled)
